(19) United States
(12) Patent Application Publication
(10) Pub. No.: US 2022/0378922 A1
(43) Pub. Date: Dec. 1, 2022

(54) Cannabinoid Conjugate Molecules

(71) Applicant: Diverse Biotech, Inc., Orlando, FL (US)

(72) Inventors: Paul Hershberger, Orlando, FL (US); Philip Arlen, Orlando, FL (US)

(73) Assignee: Diverse Biotech, Inc., Orlando, FL (US)

(21) Appl. No.: 17/622,419

(22) PCT Filed: Jun. 24, 2020
(86) PCT No.: PCT/US20/39241

§ 371 Date: Dec. 23, 2021

Related U.S. Application Data
(60) Provisional application No. 62/960,068, filed on Jan. 12, 2020.
(60) Provisional application No. 62/899,675, filed on Sep. 12, 2019.
(60) Provisional application No. 62/865,680, filed on Jun. 24, 2019.

(51) Int. Cl. A61K 47/55; A61K 47/54; A61K 45/06

(52) U.S. Cl. CPC A61K 47/55; A61K 47/545; A61K 45/06; A61K 47/542

(57) ABSTRACT

This disclosure provides multifunctional conjugate molecules in which at least one therapeutic agent is covalently attached to a cannabinoid. The disclosed conjugate molecules are designed to deliver therapeutic benefits of both components, with release of the cannabinoid upon binding of the therapeutic agent component to its target conveying further therapeutic benefits, and can be used to treat cancer, glaucoma, confusion or dementia, and other disorders.

Each reference cited in this disclosure is incorporated by reference herein in its entirety.

### TECHNICAL FIELD

This disclosure relates generally to multifunctional therapeutics.

### DETAILED DESCRIPTION

This disclosure describes multifunctional conjugate molecules comprising a therapeutic agent component covalently attached to a cannabinoid component.

In contrast to traditional prodrugs, the disclosed conjugate molecules are designed to deliver more than one therapeutic benefit via more than one mechanism of action; this is achieved when the covalent binding of the therapeutic agent component to its target enables the release of the cannabinoid at or near the site of the therapeutic agent's action, which can then effect a second therapeutic benefit. That is, these conjugate molecules are designed to deliver the therapeutic benefits of each of their components.

For example, the formation of reactive oxygen species (ROS) is a by-product of the normal process of respiration in an oxygen-rich environment (Storz & Imlay, Curr. Opin. Microbiol. 2, 188-94, 1999). There is significant evidence in the literature for the role endogenous ROS plays in mutagenesis, as well as its contribution to the mutational burden experienced by microbes during periods of oxidative stress (reviewed in Dwyer et al., Curr. Opin. Microbiol. 12, 482-89, 2009). In fact, bacteria have evolved several enzymatic mechanisms to combat ROS toxicity (Imlay, Ann. Rev. Biochem. 77, 755-76, 2008).

ROS are generated intracellularly and include superoxide (O₂.⁻)hydrogen peroxide (H₂O₂), and highly-destructive hydroxy radicals (OH.). The species O₂.⁻ and H₂O₂ can be enzymatically eradicated by the activity of superoxide dismutases and catalases/peroxidases, respectively.

Excess intracellular levels of ROS cause damage to proteins, nucleic acids, lipids, membranes, and organelles, which can lead to activation of cell death processes such as apoptosis. Apoptosis is a tightly regulated and highly conserved process of cell death during which a cell undergoes self-destruction (Kerr et al., Br. J. Cancer 26, 239-57, 1972). Apoptosis can be triggered by a variety of extrinsic and intrinsic signals, including ROS (reviewed in Redza-Dutordoir & Averill-Bates, Biochem. Biophys. Acta 1863, 2977-92, 2016). Exposure to xenobiotics such as antibiotics and chemotherapeutic drugs can also trigger apoptosis, and is often mediated by ROS.

Cannabinoids have demonstrated their ability to promote ROS production. Cannabidiol (CBD) is a non-toxic and non-psychoactive cannabinoid that has been shown to have anti-tumor activity in multiple cancer types (Massi et al., J. Pharmacol. Exp. Ther. 308, 838-45, e-pub 2003). Activation of the endogenous cannabinoid type 1 (CB1) and type 2 (CB2) receptors has been shown to inhibit tumor progression (Velasco et al., Nat. Rev. Cancer 12, 436-44, 2012). CBD has been reported to inhibit human GBM viability in culture, an effect that was reversed in the presence of the ROS scavenger α-tocopherol/vitamin E (Velasco et al., 2012).

CBD-dependent production of ROS has been shown to accompany a reduction in glutathione (Massi et al., Cell. Mol. Sci. 63, 2057-66, 2006), an important anti-oxidant that prevents damage to cellular components by ROS. The source of CBD-dependent stress in part originated in the mitochondria and led to activation of multiple caspases involved in intrinsic and extrinsic pathways of apoptosis. Further studies analyzing CBD-treated GBM tumor tissue revealed that inhibition of lipoxygenase signaling played a role in CBD anti-tumor activity (McAllister et al., J. Neuroimmune Pharmacol. 10, 255-67, 2015). In addition, the indirect modulation of the endocannabinoid system by CBD may be attributed to the observed anti-tumor activity.

Cannabigerol (CBG) is another non-psychotropic cannabinoid that interacts with specific targets involved in carcinogenesis and has shown potent anti-tumor activity (Guindon & Hohmann, Br. J. Pharmacol. 163, 1447-63, 2011). Mechanistically, CBG, similar to CBD, appears to influence the inflammatory microenvironment that is important in the initiation and progression of cancer (Mantovani et al., Nature 454, 436-44, 2008; Solinas et al., Cancer Metastasis Rev. 29, 243-48, 2010). Moreover, CBG was also able to exert pro-apoptotic effects by selectively increasing ROS production in colorectal cancer cells but not in healthy colonic cells (Borrelli et al., Carcinogenesis 35, 2787-97, 2014).

Conjugate Molecules

Conjugate molecules comprise a therapeutic agent component covalently attached to a hydroxy group or a carboxylic acid group of a cannabinoid component. In some embodiments, the hydroxy group is an “aromatic hydroxy group;” i.e., a hydroxy group bonded directly to an aromatic hydrocarbon. In some embodiments, the hydroxy group is an “aliphatic hydroxy group;” i.e., a hydroxy group bound to a carbon that is not part of an aromatic ring.

In some embodiments, conjugate molecules contain only one therapeutic agent component. In other embodiments, when the cannabinoid component has two hydroxy groups, conjugate molecules can contain two therapeutic agent components. In such embodiments, the two therapeutic agent components can be the same or different. Also independently, the two hydroxy groups can be aliphatic or the two hydroxy groups can be aromatic, or a first hydroxy group can be aliphatic and a second hydroxy group can be aromatic.

Conjugate molecules can have one or more centers of asymmetry and can therefore be prepared either as a mixture of isomers (e.g., a racemic or diasteromeric mixture) or in an enantiomerically or diasteromerically pure form. Such forms include, but are not limited to, diastereomers, enantiomers, and atropisomers. Conjugate molecules can also include alkenes and can therefore be prepared either as a mixture of double bond isomers or independently as either an E or Z isomer. Isotopic variants of conjugate molecules can also be prepared.

Conjugate molecules can form salts. “Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth metal ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Further examples of pharmaceutically acceptable salts include those listed in Berge et al., Pharmaceutical Salts, J. Pharm. Sci. 1977 January; 66(1):1-19.

### Definitions

The following definitions apply to the descriptions of the therapeutic agent components below and to the descriptions of “Group One Substituents” and “Group Two Substituents.”

“C1-C12 linear or branched alkyl” means “each of methyl, ethyl, C3, C4, C5, C6, C7, C8, C9, C10, C11, and C12 linear alkyl and C3, C4, C5, C6, C7, C8, C9, C10, C11, and C12 branched alkyl.”

“C2-C12 linear or branched alkenyl” means “each of C2, C3, C4, C5, C6, C7, C8, C9, C10, C11, and C12 linear alkenyl and C3, C4, C5, C6, C7, C8, C9, C10, C11, and C12 branched alkenyl.”

“C1-C12 linear or branched heteroalkyl” means “each of linear or branched heteroalkyl containing 1, 2, 3, 4, 5, 6, 7, 8, 9, 10, 11, or 12 carbon atoms.”

“C1-C3 linear or branched alkyl” means “methyl, ethyl, propyl, and isopropyl.”

“C1-C8 linear or branched alkyl” means “methyl, ethyl, C3, C4, C5, C6, C7, and C8 linear alkyl and C3, C4, C5, C6, C7, and C8 branched alkyl.”

“C1-C3 linear or branched heteroalkyl” means “a linear or branched heteroalkyl containing 1, 2, or 3 carbon atoms.”

“C1-C8 linear or branched heteroalkyl” means “each of a C1, C2, C3, C4, C5, C6, C7, and C8 linear heteroalkyl and C1, C2, C3, C4, C5, C6, C7, and C8 branched heteroalkyl.”

“C1-C12 linear or branched heteroalkyl” means each of a C1, C2, C3, C4, C5, C6, C7, C8, C9, C10, C11, and C12 linear heteroalkyl and C1, C2, C3, C4, C5, C6, C7, C8, C9, C10, C11, and C12 branched heteroalkyl.”

“C1-C6 linear or branched alkoxyl” means “a linear or branched alkoxyl containing 1, 2, 3, 4, 5, or C carbon atoms.”

“C1-C6 linear or branched alkylamino” means “a linear or branched alkylamino containing 1, 2, 3, 4, 5, or 6 carbon atoms.”

“C1-C6 linear or branched dialkylamino” means “each linear or branched dialkylamino in which each alkyl independently contains 1, 2, 3, 4, 5, or 6 carbon atoms.”

“6-10-membered aromatic” means “each of a 6-, 7-, 8-, 9-, and 10-membered aromatic.”

“5- to 10-membered heteroaromatic” means “each of a 6-, 7-, 8-, 9-, and 10-membered heteroaromatic.”

“3- to 9-membered cycloheteroalkyl” means “each of a 3-, 4-, 5-, 6-, 7-, 8-, and 9-membered cycloheteroalkyl.

“C3-C6 cycloalkyl” means “C3, C4, C5, and C6 cycloalkyl.”

“Halide” means “Cl, Br, and I.”

“Group One Substituents” is a group of substituents consisting of:
(a) —OH;
(b) —NH2;
(c) ═O;
(d) ═S;
(e) ═NR7, where R7 is H or is C1-C3 linear or branched alkyl or C1-C3 linear or branched heteroalkyl comprising an O, N, or S atom;
(f) —C(O)OR4, wherein R4 is H or C1-C3 linear or branched alkyl;
(g) —C(O)NR5R6, wherein R5 and R6 independently are H or C1-C6 linear or branched alkyl;
(h) halide;
(i) C1-C6 linear or branched alkoxyl;
(j) C1-C6 linear or branched alkylamino;
(k) C1-C6 linear or branched dialkylamino;
(l) 6- to 10-membered aromatic, optionally substituted with 1, 2, 3, or 4 substituents independently selected from
   (i) phenyl;
   (ii) halide;
   (iii) cyano;
   (iv) C1-C6 linear or branched alkyl, optionally substituted with
      (1) 1, 2, 3, 4, 5, 6, 7, 8, or 9 fluorine atoms; and/or
      (2) 1, 2, or 3 substituents independently selected from the Group Two Substituents; and
   (v) C1-C6 linear or branched heteroalkyl containing 1, 2, or 3 atoms independently selected from O, N, and S and optionally substituted with
      (1) 1, 2, 3, 4, 5, 6, 7, 8, or 9 fluorine atoms; and/or
      (2) 1, 2, or 3 substituents independently selected from the Group Two Substituents;
(m) 5- to 10-membered heteroaromatic, optionally substituted with 1, 2, 3, or 4 substituents independently selected from
   (i) phenyl;
   (ii) halide;
   (iii) cyano;
   (iv) C1-C6 linear or branched alkyl, optionally substituted with
      (1) 1, 2, 3, 4, 5, 6, 7, 8, or 9 fluorine atoms; and/or
      (2) 1, 2, or 3 substituents independently selected from the Group Two Substituents; and
   (v) C1-C6 linear or branched heteroalkyl containing 1, 2, or 3 atoms independently selected from O, N, and S and optionally substituted with
      (1) 1, 2, 3, 4, 5, 6, 7, 8, or 9 fluorine atoms; and/or
      (2) 1, 2, or 3 substituents independently selected from the Group Two Substituents;
(n) 3- to 9-membered cycloheteroalkyl having 1, 2, or 3 heteroatoms independently selected from O, N, and S, optionally substituted with 1, 2, 3, or 4 substituents independently selected from
   (i) phenyl;
   (ii) halide;
   (iii) cyano;
   (iv) C1-C6 linear or branched alkyl, optionally substituted with
      (1) 1, 2, 3, 4, 5, 6, 7, 8, or 9 fluorine atoms; and/or
      (2) 1, 2, or 3 substituents independently selected from the Group Two Substituents; and
   (v) C1-C6 linear or branched heteroalkyl containing 1, 2, or 3 atoms independently selected from O, N, and S and optionally substituted with
      (1) 1, 2, 3, 4, 5, 6, 7, 8, or 9 fluorine atoms; and/or
      (2) 1, 2, or 3 substituents independently selected from the Group Two Substituents; and
(o) C3-C6 cycloalkyl, optionally substituted with 1, 2, 3, or 4 substituents independently selected from
   (i) phenyl;
   (ii) halide;
   (iii) cyano;
   (iv) C1-C6 linear or branched alkyl, optionally substituted with
      (1) 1, 2, 3, 4, 5, 6, 7, 8, or 9 fluorine atoms; and/or
      (2) 1, 2, or 3 substituents independently selected from the Group Two Substituents; and
   (v) C1-C6 linear or branched heteroalkyl containing 1, 2, or 3 atoms independently selected from O, N, and S and optionally substituted with
      (1) 1, 2, 3, 4, 5, 6, 7, 8, or 9 fluorine atoms; and/or
      (2) 1, 2, or 3 substituents independently selected from the Group Two Substituents.

“Group Two Substituents” is a group of substituents consisting of:
(a) —OH;
(b) —NH2;
(c) ═O;
(d) ═S;
(e) ═NR7, where R7 is H or is C1-C3 linear or branched alkyl or C1-C3 linear or branched heteroalkyl comprising an O, N, or S atom;
(f) —C(O)OR4, wherein R4 is H or C1-C3 linear or branched alkyl;
(g) —C(O)NR5R6, wherein R5 and R6 independently are H or C1-C6 linear or branched alkyl;
(h) halide;
(i) cyano;
(j) trifluoromethyl;
(k) C1-C6 linear or branched alkoxyl;
(l) C1-C6 linear or branched alkylamino;
(m) C1-C6 linear or branched dialkylamino;
(n) 6- to 10-membered aromatic; and
(o) 5- to 10-membered heteroaromatic comprising 1, 2, 3, 4, 5, or 6 heteroatoms independently selected from O, N, and S.

The definitions above apply to the descriptions that follow. For example, the phrase “R₄ is H or C1-C3 linear or branched alkyl” should be read as describing each of five sets of embodiments in which R₄ is H, R₄ is methyl, R₄ is ethyl, R₄ is propyl, and R₄ is isopropyl.

Therapeutic Agent Component(s)

A “therapeutic agent component” as used in this disclosure is a therapeutic moiety or portion of a therapeutic agent that is present in a conjugate molecule and covalently attached to a cannabinoid. A number of therapeutic agents can be used to provide a therapeutic agent component of a conjugate molecule.

Michael Acceptors

In some embodiments, the therapeutic agent component is a Michael acceptor component. Examples of how a cannabinoid could be released from a conjugate molecule upon binding of a Michael Acceptor to a target are shown below:

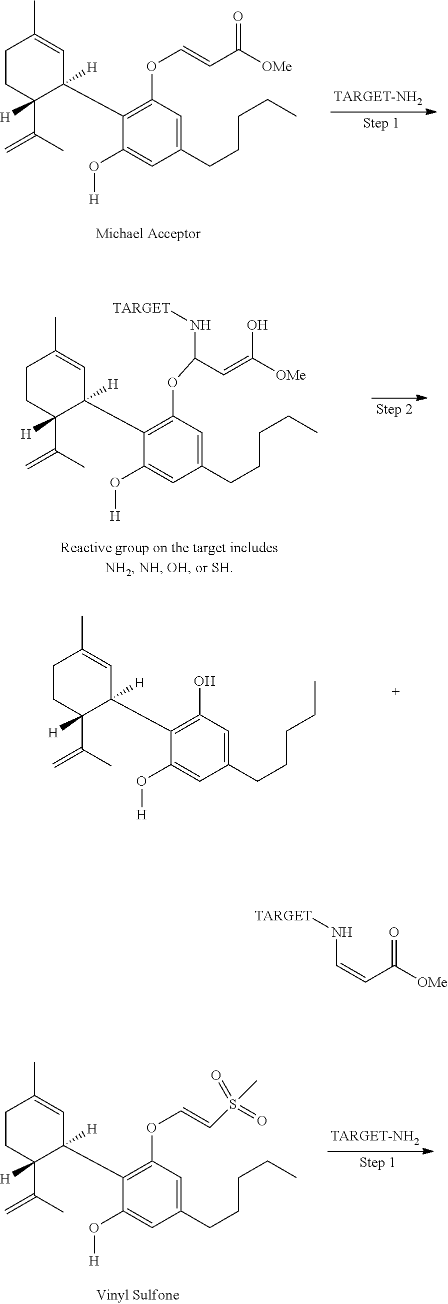

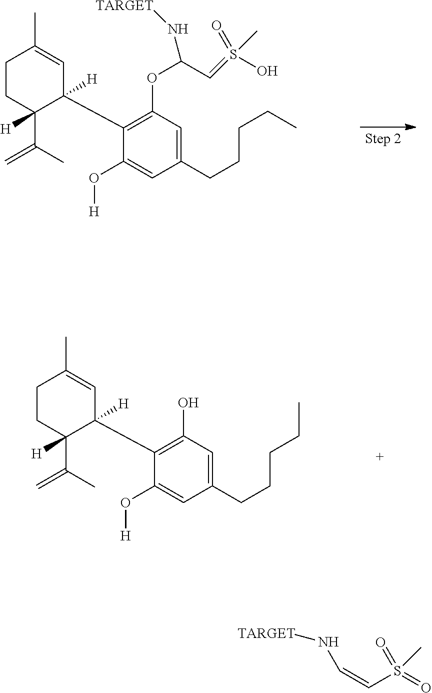

Michael acceptor components of a conjugate molecule have one of the following structures:

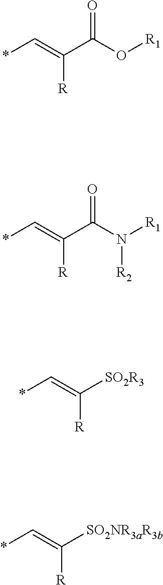

in which * indicates a site of covalent attachment to a hydroxy or carboxylic acid group of the cannabinoid component and in which

R is selected from the group consisting of:
(a) H;
(b) C1-C8 linear or branched alkyl, optionally substituted with
   (1) 1, 2, 3, 4, 5, 6, 7, 8, or 9 fluorine atoms; and/or
   (2) 1, 2, or 3 substituents independently selected from the Group One Substituents;
(c) C1-C8 linear or branched heteroalkyl containing 1, 2, or 3 heteroatoms independently selected from O, N, and S and optionally substituted with
   (1) 1, 2, 3, 4, 5, 6, 7, 8, or 9 fluorine atoms; and/or
   (2) 1, 2, or 3 substituents independently selected from the Group One Substituents;
(d) phenyl, optionally substituted with 1, 2, or 3 substituents independently selected from the group consisting of:
   (1) C1-C6 linear or branched alkyl, optionally substituted with
      (i) 1, 2, 3, 4, 5, or 6 fluorine atoms; and/or
      (ii) 1 or 2 substituents independently selected from the Group Two Substituents; and
   (2) C1-C6 linear or branched heteroalkyl containing 1 or 2 heteroatoms independently selected from O, N, and S and optionally substituted with
      (i) 1, 2, 3, 4, 5, or 6 fluorine atoms; and/or
      (ii) 1 or 2 substituents independently selected from the Group One Substituents;
(e) a 6- to 10-membered aromatic, optionally substituted with 1, 2, 3, or 4 substituents independently selected from the group consisting of:
   (1) phenyl;
   (2) halide;
   (3) cyano;
   (4) C1-C6 linear or branched alkyl, optionally substituted with
      (i) 1, 2, 3, 4, 5, 6, 7, 8, or 9 fluorine atoms; and/or
      (ii) 1, 2, or 3 substituents independently selected from the Group Two Substituents, and
   (5) C1-C6 linear or branched heteroalkyl containing 1, 2, or 3 atoms independently selected from O, N, and S and optionally substituted with
      (i) 1, 2, 3, 4, 5, 6, 7, 8, or 9 fluorine atoms; and/or
      (ii) 1, 2, or 3 substituents independently selected from the Group Two Substituents;
(f) 5- to 10-membered heteroaromatic comprising 1, 2, 3, 4, 5, or 6 heteroatoms independently selected from O, N, and S and optionally substituted with 1, 2, 3, or 4 substituents independently selected from
   (1) phenyl;
   (2) halide;
   (3) cyano;
   (4) trifluoromethyl;
   (5) C1-C6 linear or branched alkyl optionally substituted with
      (i) 1, 2, 3, 4, 5, 6, 7, 8, or 9 fluorine atoms; and/or
      (ii) 1, 2, or 3 substituents independently selected from the Group Two Substituents; and
   (6) C1-C6 linear or branched heteroalkyl containing 1, 2, or 3 atoms independently selected from O, N, and S and optionally substituted with
      (i) 1, 2, 3, 4, 5, 6, 7, 8, or 9 fluorine atoms; and/or
      (ii) 1, 2, or 3 substituents independently selected from the Group Two Substituents;
(g)

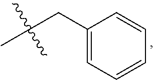 [SMILES: *Cc1ccccc1]

optionally substituted with 1, 2, or 3 substituents independently selected from the group consisting of:
   (1) C1-C6 linear or branched alkyl, optionally substituted with
      (i) 1, 2, 3, 4, 5, or 6 fluorine atoms; and/or
      (ii) 1, 2, or 3 substituents independently selected from the Group Two Substituents;
(h) 3- to 9-membered cycloheteroalkyl having 1, 2, or 3 heteroatoms independently selected from O, N, and S and optionally substituted with 1, 2, or 3 substituents independently selected from the group consisting of:
   (1) C1-C6 linear or branched alkyl, optionally substituted with
      (i) 1, 2, 3, 4, 5, 6, 7, 8, or 9 fluorine atoms; and/or
      (ii) 1, 2, or 3 substituents independently selected from the Group Two Substituents,
   (2) C1-C6 linear or branched heteroalkyl, optionally substituted with
      (i) 1, 2, 3, 4, 5, 6, 7, 8, or 9 fluorine atoms and/or
      (ii) 1, 2, or 3 substituents independently selected from the Group Two Substituents,
   (3) phenyl, optionally substituted with 1, 2, or 3 substituents independently selected from the Group Two Substituents, and
   (4) 5- to 10-membered heteroaromatic, optionally substituted with 1, 2, or 3 substituents independently selected from the Group Two Substituents; and
(i) C3-C6 cycloalkyl, optionally substituted with 1, 2, or 3 substituents independently selected from:
   (1) C1-C6 linear or branched alkyl, optionally substituted with
      (i) 1, 2, 3, 4, 5, 6, 7, 8, or 9 fluorine atoms; and/or
      (ii) 1, 2, or 3 substituents independently selected from the Group Two Substituents,
   (2) C1-C6 linear or branched heteroalkyl, optionally substituted with
      (i) 1, 2, 3, 4, 5, 6, 7, 8, or 9 fluorine atoms; and/or
      (ii) 1, 2, or 3 substituents independently selected from the Group Two Substituents,
   (3) phenyl, optionally substituted with 1, 2, or 3 substituents independently selected from Group Two Substituents; and
   (4) 5- to 10-membered heteroaromatic, optionally substituted with 1, 2, or 3 substituents independently selected from the Group Two Substituents; and

R₁ and R₂ independently are selected from the group consisting of:
(a) C1-C12 linear or branched alkyl, optionally substituted with
   (i) 1, 2, 3, 4, 5, 6, 7, 8, or 9 fluorine atoms; and/or
   (ii) 1, 2, or 3 substituents selected from the Group One Substituents;
(b) C2-C12 linear or branched alkenyl, optionally substituted with
   (i) 1, 2, 3, 4, 5, 6, 7, 8, or 9 fluorine atoms; and/or
   (ii) 1, 2, or 3 substituents selected from the Group One Substituents;
(c) C1-C12 linear or branched heteroalkyl containing 1, 2, 3, or 4 heteroatoms independently selected from O, N, and S, optionally substituted with
   (i) 1, 2, 3, 4, 5, 6, 7, 8, or 9 fluorine atoms; and/or
   (ii) 1, 2, or 3 substituents selected from the Group One Substituents; and
(d) R; OR

R₁ and R₂, together with the atom to which they are attached, form a 3- to 9-membered cycloheteroalkyl having 1, 2, 3, or 4 heteroatoms independently selected from O, S, and N, wherein the cycloheteroalkyl optionally is substituted with 1, 2, or 3 substituents independently selected from, C1-C6 linear or branched alkyl optionally substituted with 1, 2, 3, 4, 5, 6, 7, 8, or 9 fluorine atoms, C1-C6 linear or branched heteroalkyl optionally substituted with 1, 2, 3, 4, 5, 6, 7, 8, or 9 fluorine atoms, phenyl optionally substituted with 1, 2, or 3 substituents independently selected from Group Two Substituents, or 5- to 10-membered heteroaromatic optionally substituted with 1, 2, or 3 independently selected from Group Two Substituents;

R₃, R₃ₐ, and R_{3b} independently are selected from
(a) C1-C8 linear or branched alkyl, optionally substituted with
   (i) 1, 2, 3, 4, 5, 6, 7, 8, or 9 fluorine atoms; and/or
   (ii) 1, 2, or 3 substituents independently selected from the Group One Substituents; or
(b) phenyl, optionally substituted with up to three substituents independently selected from the group consisting of C1-C6 linear or branched alkyl, optionally substituted with
   (i) 1, 2, 3, 4, 5, or 6 fluorine atoms; and/or
   (ii) 1 or 2 substituents independently selected from the Group Two Substituents; and
      R₆ and R₇ independently are R.

Examples of therapeutic agents that can be used to provide a Michael acceptor component include, but are not limited to:

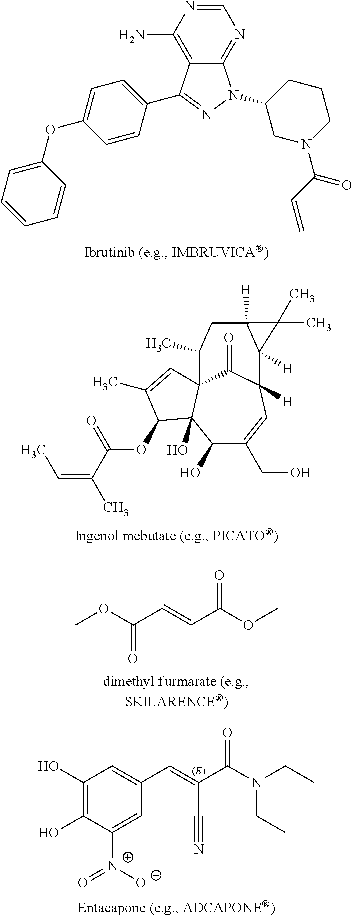

Examples of Michael acceptor components include, but are not limited to:

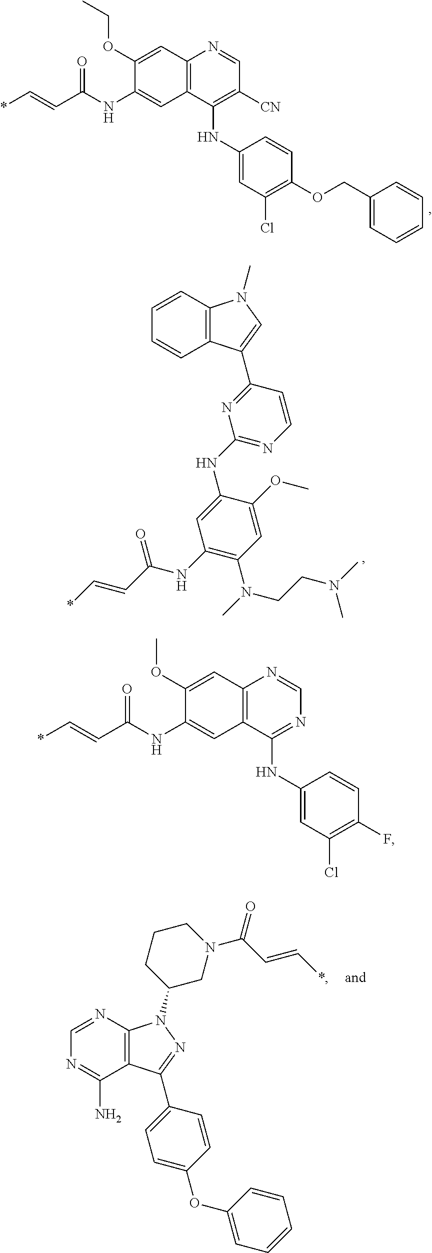

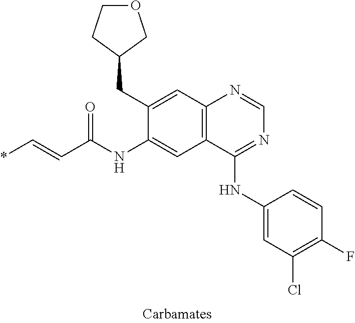 [SMILES: */C=C/C(=O)Nc1cc2c(Nc3ccc(F)c(Cl)c3)ncnc2cc1C[C@H]1CCOC1]

In some embodiments, the therapeutic agent component is a carbamate component. Examples of how a cannabinoid can be released from a conjugate molecule upon binding of a carbamate to a target are shown below:

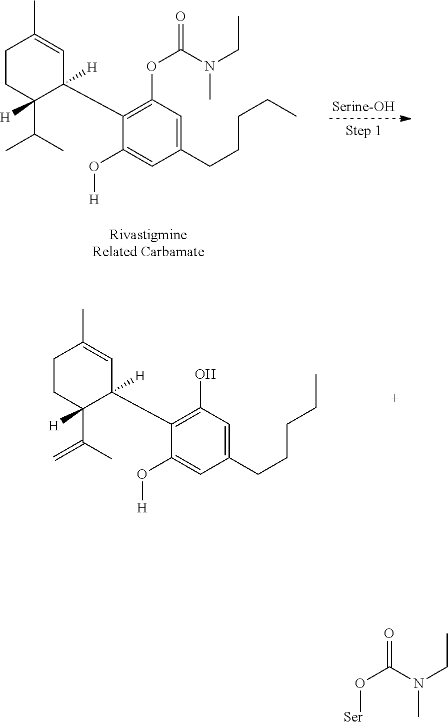

Carbamate components of a conjugate molecule have the following structure:

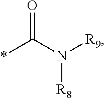

in which * indicates a site of covalent attachment to a hydroxy or carboxylic acid group of the cannabinoid component and in which R₈ and R₉ independently are selected from H, CH₃, and CH₂CH₃.

Hydroxyurea Derivatives

In some embodiments, the therapeutic agent component is a hydroxyurea derivative. Hydroxyurea is a ribonucleotide reductase inhibitor. An example of how a cannabinoid may be released from a conjugate molecule upon the hydroxyurea first functioning therapeutically as a radical scavenger with generation of nitric oxide and CO₂ is shown below. A more detailed description of the mechanism of action for the parent hydroxyurea molecule with conversion to NO, CO₂, and NH₃ is reported (sciencedirect.com/topics/chemistry/hydroxyurea).

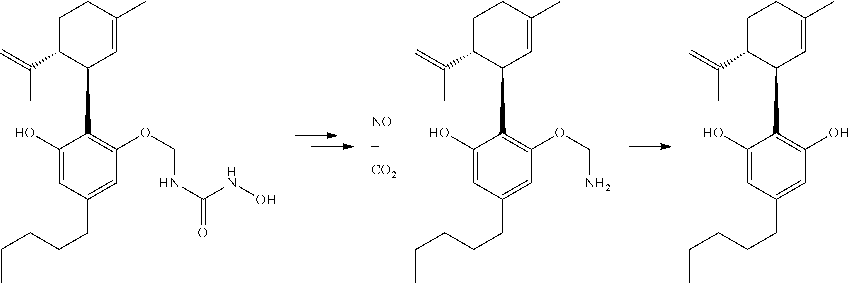 [SMILES: C=C(C)[C@@H]1CCC(C)=C[C@H]1c1c(O)cc(CCCCC)cc1OCNC(=O)NO ; "CO2" ; "NO" ; C=C(C)[C@@H]1CCC(C)=C[C@H]1c1c(O)cc(CCCCC)cc1OCN ; C=C(C)[C@@H]1CCC(C)=C[C@H]1c1c(O)cc(CCCCC)cc1O]

Hydroxyurea derivative components of a conjugate molecule have the structure

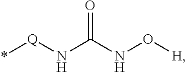

in which * indicates a site of covalent attachment to a hydroxy or carboxylic acid group of the cannabinoid component, Q is CO, CS, or CR₆R₇, and R₆ and R₇ independently are R as defined above.

Cannabinoid Component

A “cannabinoid component” as used in this disclosure is that portion of the cannabinoid that is present in the conjugate molecule and covalently attached to the therapeutic agent component, as shown in the examples below.

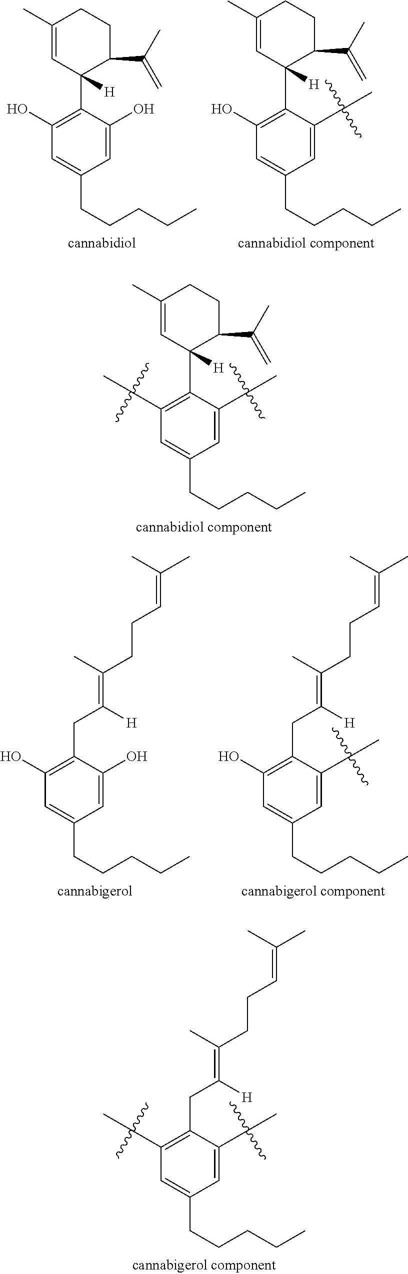 [SMILES: C=C(C)[C@@H]1CCC(C)=C[C@H]1c1c(O)cc(CCCCC)cc1O ; *c1cc(CCCCC)cc(O)c1[C@@H]1C=C(C)CC[C@H]1C(=C)C ; *c1cc(CCCCC)cc(*)c1[C@@H]1C=C(C)CC[C@H]1C(=C)C ; CCCCCc1cc(O)c(C/C=C(\C)CCC=C(C)C)c(O)c1 ; *c1cc(CCCCC)cc(O)c1C/C=C(\C)CCC=C(C)C ; *c1cc(CCCCC)cc(*)c1C/C=C(\C)CCC=C(C)C]

The cannabinoid component can be provided by any cannabinoid that contains a hydroxy or carboxylic acid group to which the therapeutic agent component can be attached. The cannabinoid can be a naturally occurring molecule, either isolated or synthesized, or a modified version of a naturally occurring molecule. See, for example, Morales et al., Frontiers in Pharmacology June 2017 review, 1-18.

Examples of cannabinoids include, but are not limited to, cannabigerols, cannabichromenes, cannabidiols, tetrahydrocannabinols, cannabicyclols, cannabielsoins, cannabinols, cannabinodiols, cannabitriols, dehydrocannabifurans, cannabifurans, cannabichromanons, and cannabiripsols.

Examples of cannabigerols include cannabigerolic acid (CBGA), cannabigerolic acid monomethylether (CBGAM), cannabigerol (CBG), cannabigerol monomethyleither (CBGM), cannabigerovarinic acid (CBGVA), and cannabigerovarin (CBGV).

Examples of cannabichromenes include cannabichromenic acid (CBC), cannabichromene (CBC), cannabichromevarinic acid (CBCVA), and cannabichromevarin (CBCV).

Examples of cannabidiols include cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), and cannabidiorcol (CBD-C₁).

Examples of tetrahydrocannabinols include Δ-9-tetrahydrocannabinolic acid A (THCA-A), Δ-9-tetrahydrocannabinolic acid B (THCA-B), Δ-9-tetrahydrocannabinol (THC), Δ-9-tetrahydrocannabinolic acid-C₄ (THCA-C₄), Δ-9-tetrahydrocannabinol-C₄ (THC-C₄), Δ-9-tetrahydrocannabivarinic acid (THCVA), Δ-9-tetrahydrocannabivarin (THCV), Δ-9-tetrahydrocannabiorcolic acid (THCA-C₁), Δ-9-tetrahydrocannabiorcol (THC-C₁), Δ-7-cis-tetrahydrocannabivarin, Δ-8-tetrahydrocannabinolic acid (Δ⁸-THCA), and Δ-8-tetrahydrocannabinol (Δ⁸-THC).

Examples of cannabicyclols include cannabicyclolic acid (CBLA), cannabicyclol (CBL), and cannabicyclovarin (CBLV).

Examples of cannabielsoins include cannabielsoic acid A (CBEA-A), cannabielsoic acid B (CBEA-B), and cannabielsoin (CBE).

Examples of cannabinols and cannabinodiols include cannabinolic acid (CBNA), cannabinol (CBN), cannabinol-C4 (CBN-C₄), cannabivarin (CBV), cannabinol-C2 (CBN-C₂), cannabiorcol (CBN-C₁), cannabinodiol (CBND), and cannabinodivarin (CBVD).

Examples of cannabitriols include cannabitriol (CBT), 10-ethoxy-9-hydroxy-Δ-6a-tetrahydrocannabinol, cannabitriolvarin (CBTV), and ethoxy-cannabitriolvarin (CBTVE).

Cannabifurans include dehydrocannabifuran (DCBF) and cannabifuran (CBF).

Examples of other cannabinoids include cannabichromanon (CBCN), 10-oxo-Δ-6a-tetrahydrocannabinol (OTHC), cannabiripsol (CBR), and trihydroxy-Δ-9-tetrahydrocannabinol (triOH-THC).

In some embodiments, the cannabinoid component is provided by cannabidiol.

Conjugate Molecules Comprising Two Therapeutic Agent Components

In some embodiments, in which the cannabinoid component has at least two hydroxy groups, at least one hydroxy group and at least one carboxylic acid group, or at least two carboxylic acid groups, a second therapeutic agent component can be covalently attached to the second hydroxy group such that the conjugate molecule contains a first therapeutic agent component and a second therapeutic agent component. In conjugate molecules comprising two therapeutic agent components, the therapeutic agent components can be the same or different.

Examples of Conjugate Molecules

Examples of conjugate molecules are shown below. For simplicity, the cannabinoid component is a cannabidiol component covalently attached to a single therapeutic agent component.

Conjugate Molecules with Michael Acceptor Components

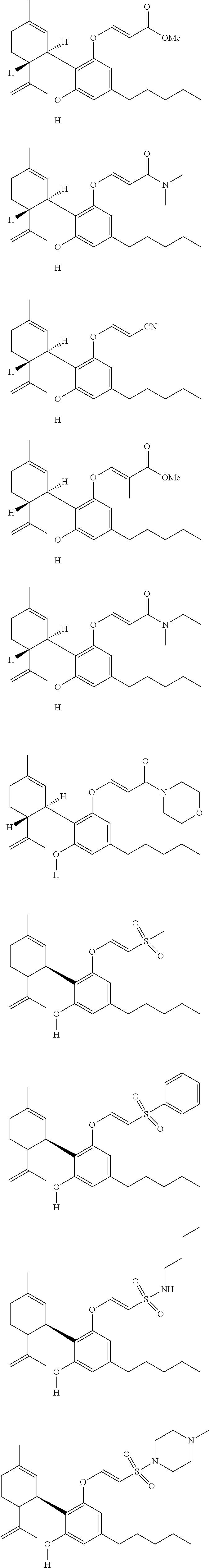

Conjugate Molecules with Carbamate Components

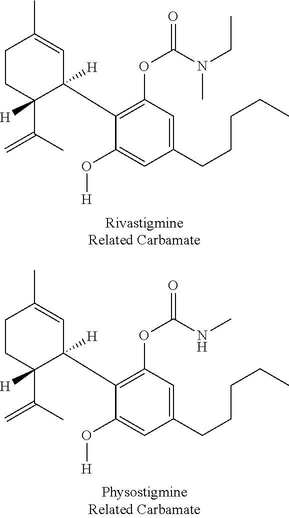 [SMILES: C=C(C)[C@@H]1CCC(C)=C[C@H]1c1c(O)cc(CCCCC)cc1OC(=O)N(C)CC ; C=C(C)[C@@H]1CCC(C)=C[C@H]1c1c(O)cc(CCCCC)cc1OC(=O)NC]

Conjugate Molecules with Hydroxyurea Components

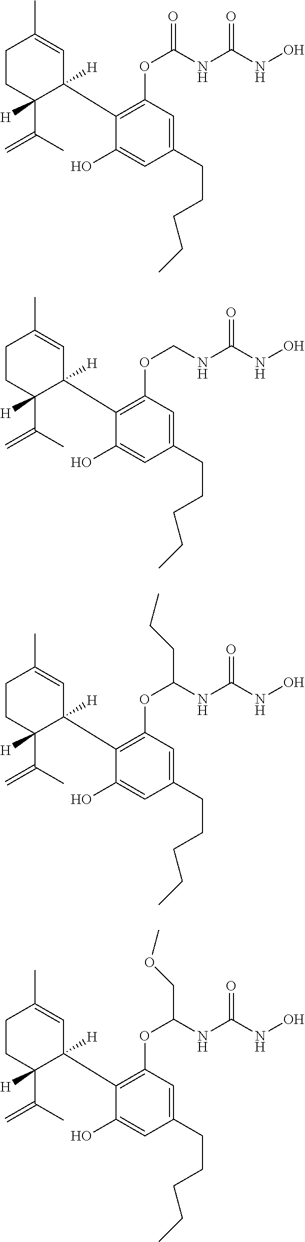

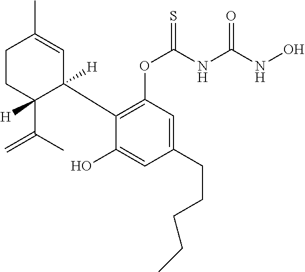 [SMILES: C=C(C)[C@@H]1CCC(C)=C[C@H]1c1c(O)cc(CCCCC)cc1OC(=S)NC(=O)NO]

Pharmaceutical Compositions, Routes of Administration, and Dosages

One or more conjugate molecules, which can be the same or different, can be provided in a pharmaceutical composition together with a pharmaceutically acceptable vehicle. The “pharmaceutically acceptable vehicle” can comprise one or more substances which do not affect the biological activities of the conjugate molecules and, when administered to a patient, do not cause an adverse reaction. Excipients, such as calcium carbonate, calcium phosphate, various sugars and types of starch, cellulose derivatives, and gelatin can be included. Pharmaceutically acceptable vehicles for liquid compositions include, but are not limited to, water, saline, polyalkylene glycols (e.g., polyethylene glycol), vegetable oils, and hydrogenated naphthalenes. Controlled release, for example, can be achieved using biocompatible, biodegradable polymers of lactide or copolymers of lactide/glycolide or polyoxyethylene/polyoxypropylene.

Methods of preparing pharmaceutical compositions are well known. Pharmaceutical compositions can be prepared as solids, semi-solids, or liquid forms, such as tablets, capsules, powders, granules, ointments, solutions, suspensions, emulsions, suppositories, injections, inhalants, gels, microspheres, aerosols, and mists. Liquid pharmaceutical compositions can be lyophilized. Lyophilized compositions can be provided in a kit with a suitable liquid, typically water for injection (WFI) for use in reconstituting the composition.

Typical administration routes include, but are not limited to, oral, topical, transdermal, inhalation, parenteral, sublingual, buccal, rectal, vaginal, and intranasal.

The dose of a pharmaceutical composition can be based on the doses typically used for the particular therapeutic agent(s) which provide the therapeutic agent component(s) of a conjugate molecule. These doses are well known in the art.

Therapeutic Methods

The disclosed conjugate molecules have a variety of therapeutic uses depending on which therapeutic agent component(s) are included in a conjugate molecule. “Treat” as used in this disclosure means reducing or inhibiting the progression of one or more symptoms of the disorder or disease for which the conjugate molecule is administered, such as inflammation or pain.

For example, a conjugate molecule having a Michael Acceptor component can be used to treat hyperproliferative disorders, including cancers. For example, treatment of cancer may include inhibiting the progression of a cancer, for example, by reducing proliferation of neoplastic or pre-neoplastic cells; destroying neoplastic or pre-neoplastic cells; or inhibiting metastasis or decreasing the size of a tumor. Cancers that can be treated include, but are not limited to, multiple myeloma (including systemic light chain amyloidosis and Waldenström's macroglobulinemia/lymphoplasmocytic lymphoma), myelodysplastic syndromes, myeloproliferative neoplasms, gastrointestinal malignancies (e.g., esophageal, esophagogastric junction, gallbladder, gastric, colon, pancreatic, hepatobiliary, anal, and rectal cancers), leukemias (e.g., acute myeloid, acute myelogenous, chronic myeloid, chronic myelogenous, acute lymphocytic, acute lymphoblastic, chronic lymphocytic, and hairy cell leukemia), Hodgkin lymphoma, non-Hodgkin's lymphomas (e.g., B-cell lymphoma, hairy cell leukemia, primary cutaneous B-cell lymphoma, and T-cell lymphoma), lung cancer (e.g., small cell and non-small cell lung cancers), basal cell carcinoma, plasmacytoma, breast cancer, bladder cancer, kidney cancer, neuroendocrine tumors, adrenal tumors, bone cancer, soft tissue sarcoma, head and neck cancer, thymoma, thymic carcinoma, cervical cancer, uterine cancers, ovarian cancer (e.g., Fallopian tube and primary peritoneal cancers), vaginal cancer, vulvar cancer, penile cancer, testicular cancer, prostate cancer, melanoma (e.g., cutaneous and uveal melanomas), non-melanoma skin cancers (e.g., basal cell skin cancer, dermatofibrosarcoma protuberans, Merkel cell carcinoma, and squamous cell skin cancer), malignant pleural mesothelioma, central nervous system (CNS) cancers (e.g., astrocytoma, oligodendroglioma, anaplastic glioma, glioblastoma, intra-cranial ependymoma, spinal ependymoma, medulloblastoma, CNS lymphoma, spinal cord tumor, meningioma, brain metastases, leptomeningeal metastases, metastatic spine tumors), and occult primary cancers (i.e., cancers of unknown origin).

Conjugate molecules described herein can be administered in conjunction with one or more other cancer therapies such as chemotherapies, immunotherapies, tumor-treating fields (TTF; e.g., OPTUNE® system), radiation therapies (XRT), and other therapies (e.g., hormones, autologous bone marrow transplants, stem cell reinfusions). “In conjunction with” includes administration together with, before, or after administration of the one or more other cancer therapies.

Chemotherapies include, but are not limited to, FOLFOX (leucovorin calcium, fluorouracil, oxaliplatin), FOLFIRI (leucovorin calcium, fluorouracil, irinotecan), FOLFIRINOX (leucovorin calcium, fluorouracil, irinotecan, oxaliplatin), irinotecan (e.g., CAMPTOSAR®), capecitabine (e.g., XELODA®, gemcitabine (e.g., GEMZAR®), paclitaxel (e.g., ABRAXANE®), dexamethasone, lenalidomide (e.g., REVLIMID®), pomalidomide (e.g., POMALYST®), cyclophosphamide, regorafenib (e.g., STIVARGA®), erlotinib (e.g., TARCEVA®), ixazomib (e.g., NINLARO®), bevacizumab (e.g., AVASTIN®), bortezomib (e.g., VELCADE®, NEOMIB®), cetuximab (e.g., ERBITUX®), daratumumab (e.g., DARZALEX®), elotumumab (e.g., EMPLICITI®), carfilzomib (e.g., KYPROLIS®), palbociclib (e.g., IBRANCE®, fulvestrant (e.g., FASLODEX®), carboplatin, cisplatin, taxol, nab paclitaxel (e.g., ABRAXANE®), 5-fluorouracil, RVD (lenalidomide, bortezomib, dexamethasone), pomolidamide (e.g., POMALYST®), temozolomide (e.g., TEMODAR®), PCV (procarbazine, lomustine, vincristine), methotrexate (e.g., TREXALL®, RASUVO®, XATMEP®), carmustine (e.g., BICNU®, GLIADEL WAFER®), etoposide (e.g., ETOPOPHOS®, TOPOSAR®), sunitinib (e.g., SUTENT®), everolimus (e.g., ZORTRESS®, AFINITOR®), rituximab (e.g., RITUXAN®, MABTHERA®), R-MPV (vincristine, procarbazine, rituximab), cytarabine (e.g., DEPOCYT®, CYTOSAR-U®), thiotepa (e.g., TEPADINA®), busulfan (e.g., BUSULFEX®, MYLERAN®), TBC (thiotepa, busulfan, cyclophosphamide), ibrutinib (e.g., IMBRUVICA®, topotecan (e.g., HYCAMTIN®), pemetrexed (e.g., ALIMTA®), vemurafenib (e.g., ZELBORAF®), cobimetinib (e.g., COTELLIC), dabrafenib (e.g., TAFINLAR®), trametinib (e.g., MEKINIST®), alectinib (e.g., ALECENSA®), lapatinib (e.g., TYKERB®), neratinib (e.g., NERLYNX®), ceritinib (e.g., ZYKADIA®), brigatinib (e.g., ALUNBRIG®), afatinib (e.g., GILOTRIF®, GIOTRIF®), gefitinib (e.g., IRESSA), osimertinib (e.g., TAGRISSO®, TAGRIX®), and crizotinib (e.g., XALKORI®).

Immunotherapies include, but are not limited to, checkpoint inhibitors, including monoclonal antibodies such as ipilimumab (e.g., YERVOY®), nivolumab (e.g., OPDIVO®), pembrolizumab (e.g., KEYTRUDA®); cytokines; cancer vaccines; and adoptive cell transfer.

In some embodiments, one or more conjugate molecules described above are administered to a patient with a cancer, including any of those cancers listed above. In some embodiments, as described below, the patient has colon cancer, rectal cancer, pancreatic cancer, multiple myeloma, or glioblastoma multiforme and the conjugate molecule(s) are administered in conjunction with an additional therapy appropriate for the particular cancer.

A conjugate molecule having a hydroxyurea component can be used to treat chronic myeloid leukemia, ovarian cancer, and squamous cell cancers of the head and neck, as well as to reduce episodes of pain and the need for blood transfusions in patients with sickle cell anemia.

A conjugate molecule having a temozolomide component can be used to treat brain cancers (e.g., astrocytoma, glioblastoma multiforme).

A conjugate molecule having a physostigmine-based carbamate component can be used to treat glaucoma and to reverse central and peripheral anticholinergia. A conjugate molecule having a rivastigmine-based carbamate component can be used to treat confusion or dementia in, for example, in patients with Alzheimer's disease or Parkinson's disease.

The disclosed conjugate molecules can be used to treat these and other disorders in the same way the therapeutic agent components of the molecules are used, and these methods are well known. An advantage of conjugate molecules, however, is that the cannabinoid can be delivered directly to the site of action of the therapeutic agent, where the released cannabinoid can provide further therapeutic benefits. The therapeutic benefits and potential benefits of cannabinoids are well known. For example, see Dzierzanowski, Cancers 11, 129-41, 2019 (oncology and palliative care); Urits et al., Pain Ther. 8, 41-51, 2019 (pain); Hillen et al., Ther. Adv. Drug Safety 10, 1-23 2019 (neuropsychiatric symptoms in dementia).

### EXAMPLES

The following procedures for synthesizing various types and classes of compounds are general representative procedures for building in the primary functionality of the compounds. The reagent system and reaction conditions may vary for any specific analog. Specific building blocks vary in accordance with the specific desired product. The procedures below show cannabidiol (CBD) as a representative cannabinoid, although other cannabinoid containing hydroxy groups may be substituted to generate alternative analogs.

### Example 1. Synthesis of Conjugate Molecules with Hydroxyurea Components

Hydroxyurea aminal linked compounds are synthesized as follows. A cannabinoid (CBD in this example) is converted to its chloromethyl derivative using previously described conditions (see Scheme below). The chloromethyl group is converted to the corresponding aminomethyl intermediate using standard tranformations, in this case by way of the azide. The aminomethyl group is converted to the isocyanate intermediate using the referenced conditions (see Scheme). Reaction of the isocyanate with hydroxylamine gives the desired product.

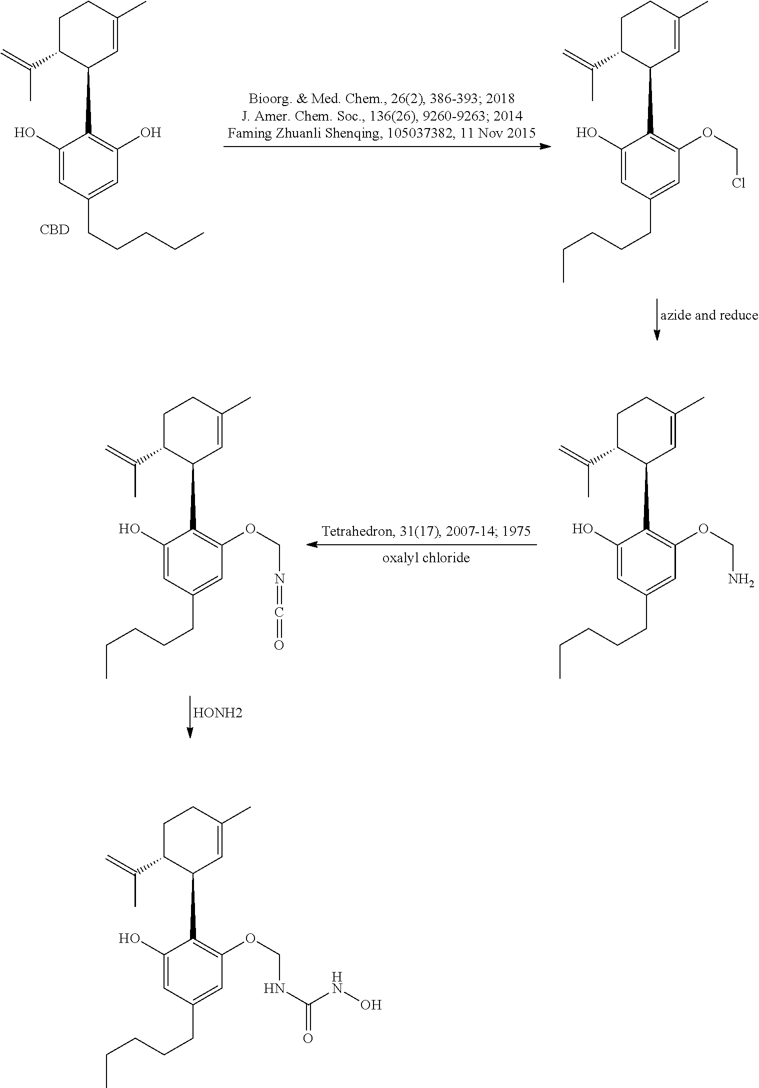

Hydroxyurea carbamate linked compounds are synthesized as follows. A cannabinoid (CBD in this example) is reacted with phosgene (or a suitable surrogate) and the adduct is converted to the carbamate intermediate using the referenced conditions (see Scheme). Conversion to the isocyanate (referenced conditions) followed by reaction with hydroxylamine gives the desired product.

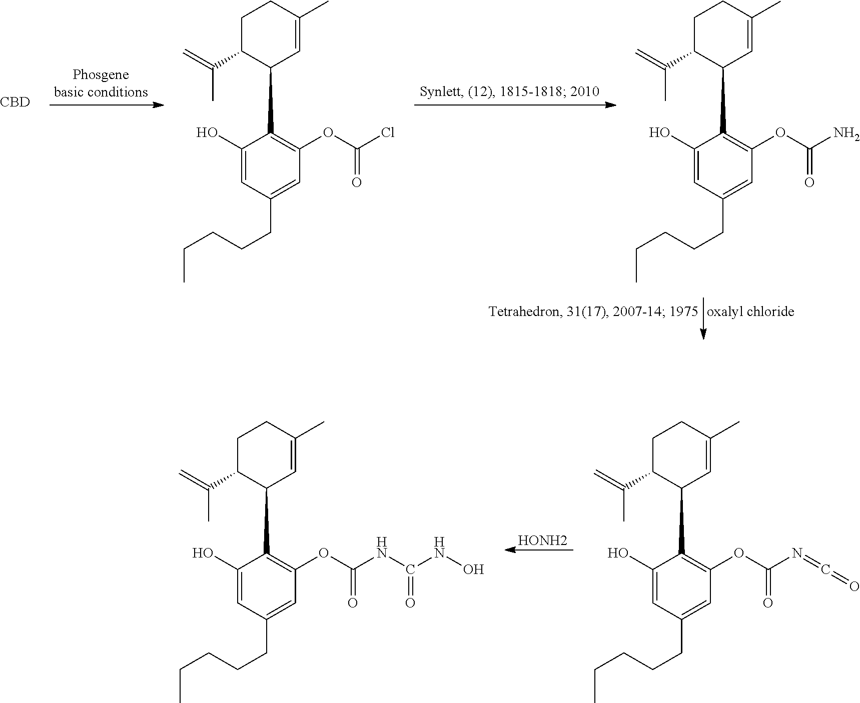 [SMILES: "CBD" ; C=C(C)[C@@H]1CCC(C)=C[C@H]1c1c(O)cc(CCCCC)cc1OC(=O)Cl ; C=C(C)[C@@H]1CCC(C)=C[C@H]1c1c(O)cc(CCCCC)cc1OC(N)=O ; C=C(C)[C@@H]1CCC(C)=C[C@H]1c1c(O)cc(CCCCC)cc1OC(=O)NC(=O)NO ; "HONH2" ; C=C(C)[C@@H]1CCC(C)=C[C@H]1c1c(O)cc(CCCCC)cc1OC(=O)N=C=O]

Hydroxyurea thiocarbamate linked compounds are synthesized as follows. A cannabinoid (CBD in this example) is reacted with thiophosgene (or a suitable surrogate) and the adduct is converted to the thiocarbamate intermediate using the referenced conditions (see Scheme). Conversion to the isocyanate (referenced conditions) followed by reaction with hydroxylamine gives the desired product.

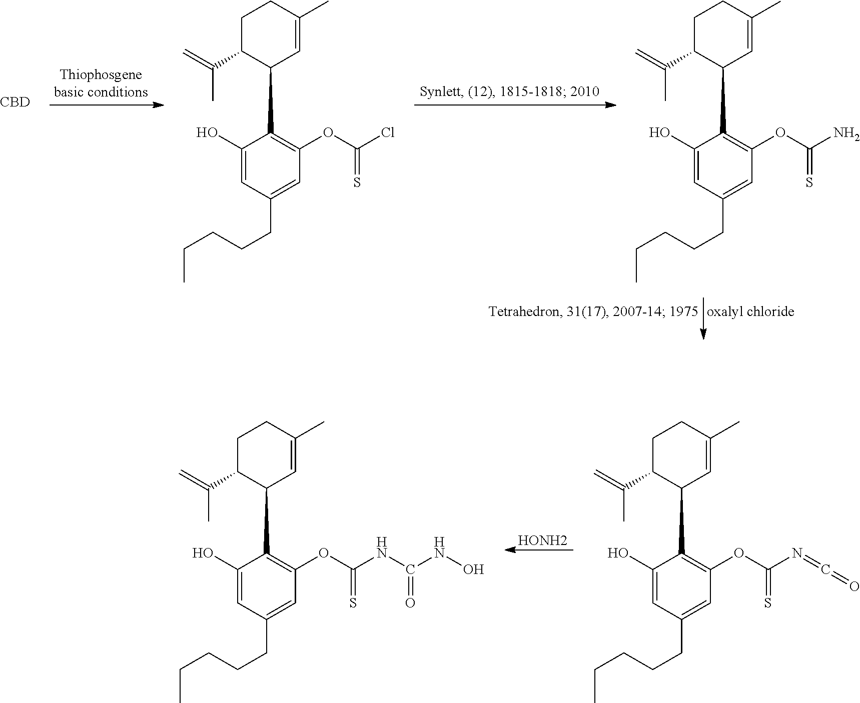 [SMILES: "CBD" ; C=C(C)[C@@H]1CCC(C)=C[C@H]1c1c(O)cc(CCCCC)cc1OC(=S)Cl ; C=C(C)[C@@H]1CCC(C)=C[C@H]1c1c(O)cc(CCCCC)cc1OC(N)=S ; C=C(C)[C@@H]1CCC(C)=C[C@H]1c1c(O)cc(CCCCC)cc1OC(=S)NC(=O)NO ; "HONH2" ; C=C(C)[C@@H]1CCC(C)=C[C@H]1c1c(O)cc(CCCCC)cc1OC(=S)N=C=O]

### Example 2. Synthesis of Conjugate Molecules with Michael Acceptor Components

Michael Acceptor amide compounds are synthesized as follows. A cannabinoid (CBD in this example) is reacted with an alkynyl ester, in this case [623-47-2] under reported conditions (see Scheme) to give the unsaturated acid intermediate. Reaction with an amine, in this case diethylamine, under standard amide bond forming conditions gives the desired product.

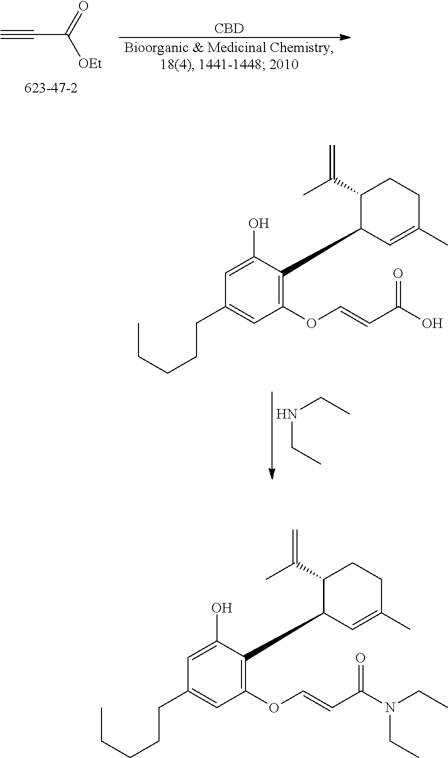

Michael Acceptor ester compounds are synthesized as follows. A cannabinoid (CBD in this example) is reacted with an alkynyl ester, in this case [623-47-2] under reported conditions (see Scheme) to give the desired product.

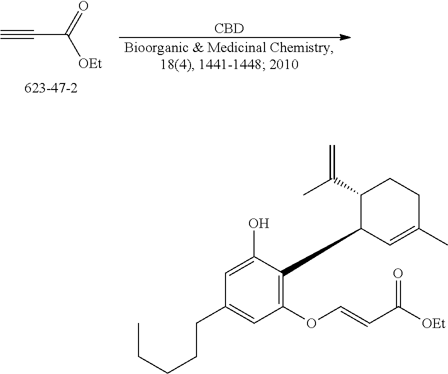

Michael Acceptor nitrile compounds are synthesized using the following referenced conditions.

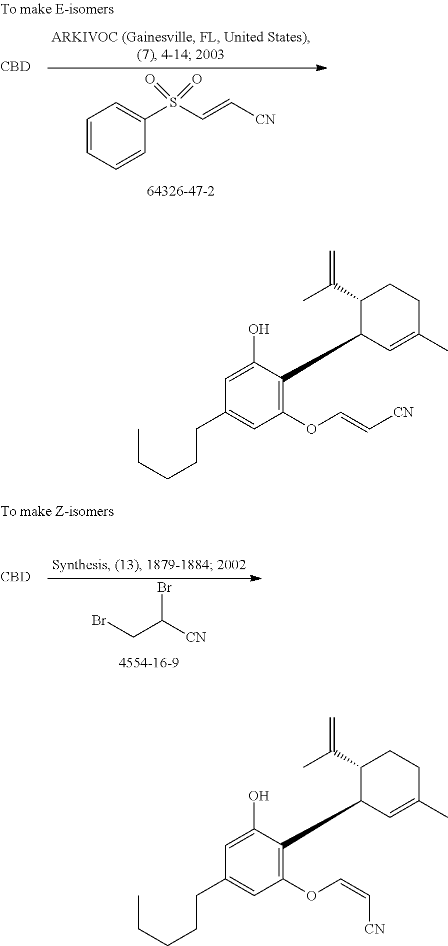 [SMILES: "CBD" ; N#C/C=C/S(=O)(=O)c1ccccc1 ; C=C(C)[C@@H]1CCC(C)=C[C@H]1c1c(O)cc(CCCCC)cc1O/C=C/C#N ; "CBD" ; N#CC(Br)CBr ; C=C(C)[C@@H]1CCC(C)=C[C@H]1c1c(O)cc(CCCCC)cc1O/C=C\C#N]

Michael Acceptor amide compounds containing a neratinib component are synthesized as follows. A cannabinoid (CBD in this example) is reacted with an alkynyl ester, in this case [623-47-2] under reported conditions (see Scheme) to give the unsaturated acid intermediate. Reaction with an amine, in this case [848139-78-6], under standard amide bond forming conditions gives the desired product.

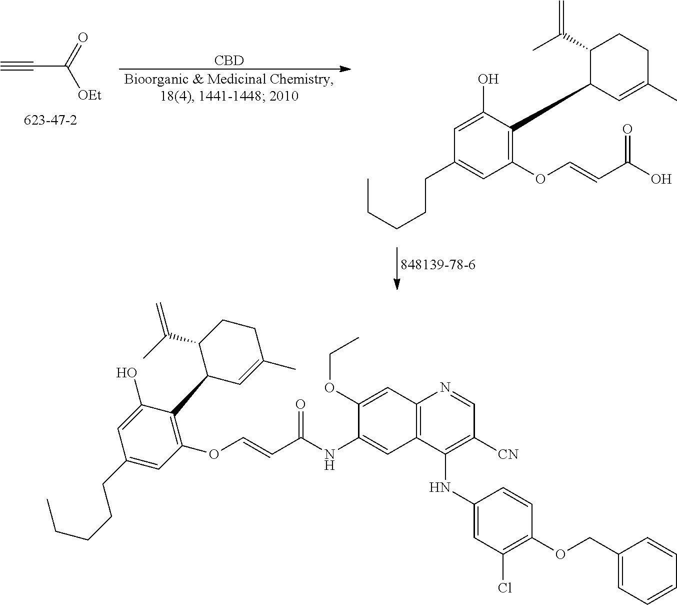

Michael Acceptor amide compounds containing a dacomitinib component are synthesized as follows. A cannabinoid (CBD in this example) is reacted with an alkynyl ester, in this case [623-47-2] under reported conditions (see Scheme) to give the unsaturated acid intermediate. Reaction with an amine, in this case [179552-75-1], under standard amide bond forming conditions gives the desired product.

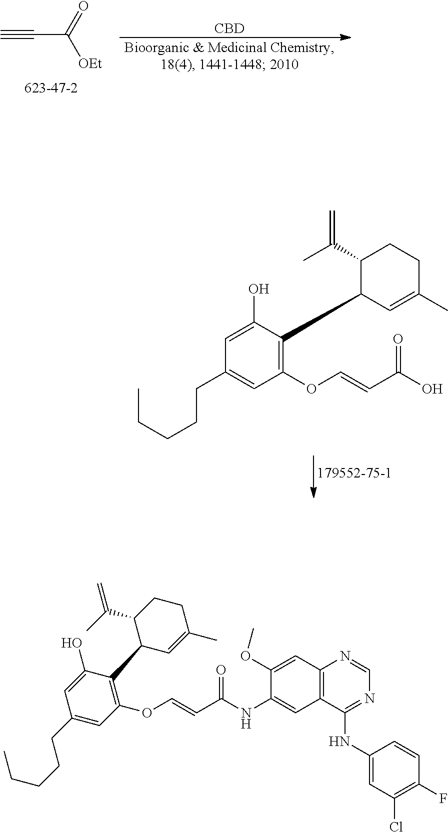

Michael Acceptor amide compounds containing a osimertinib component are synthesized as follows. A cannabinoid (CBD in this example) is reacted with an alkynyl ester, in this case [623-47-2] under reported conditions (see Scheme) to give the unsaturated acid intermediate. Reaction with an amine, in this case [1421372-66-8], under standard amide bond forming conditions gives the desired product.

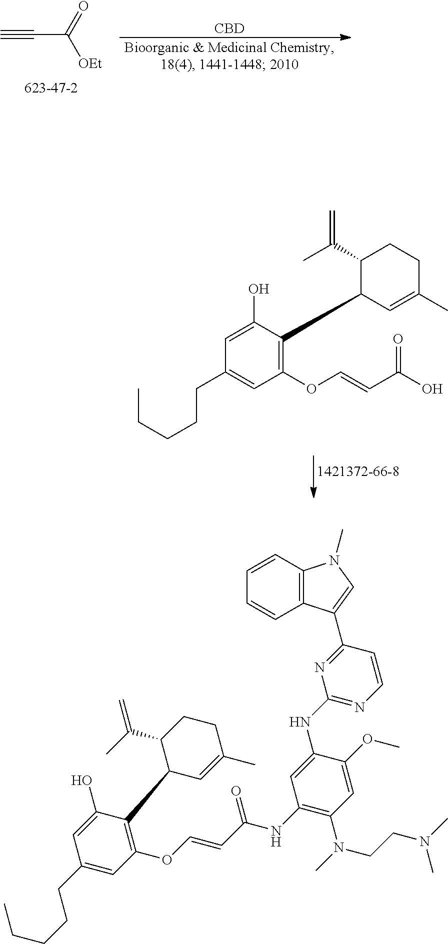

Michael Acceptor amide compounds containing an ibrutinib component are synthesized as follows. A cannabinoid (CBD in this example) is reacted with an alkynyl ester, in this case [623-47-2] under reported conditions (see Scheme) to give the unsaturated acid intermediate. Reaction with an amine, in this case [1022150-12-4], under standard amide bond forming conditions gives the desired product.

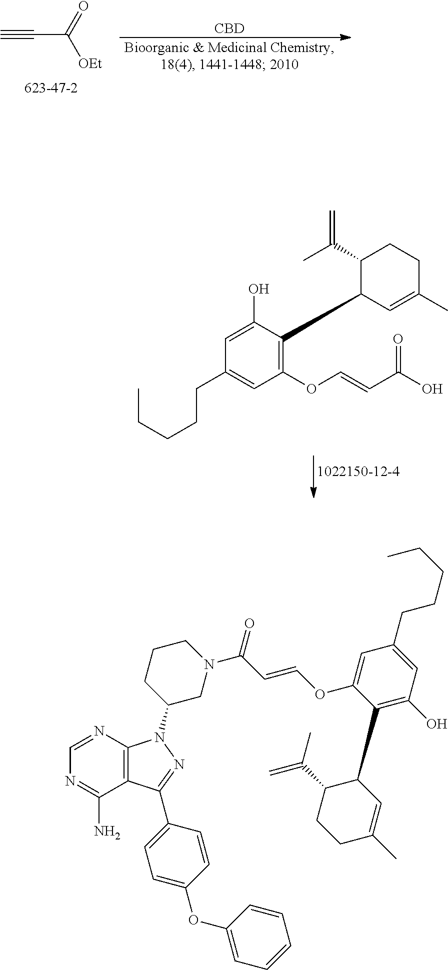

Michael Acceptor amide compounds containing an afatinib component are synthesized as follows. A cannabinoid (CBD in this example) is reacted with an alkynyl ester, in this case [623-47-2] under reported conditions (see Scheme) to give the unsaturated acid intermediate. Reaction with an amine, in this case [314771-76-1], under standard amide bond forming conditions gives the desired product.

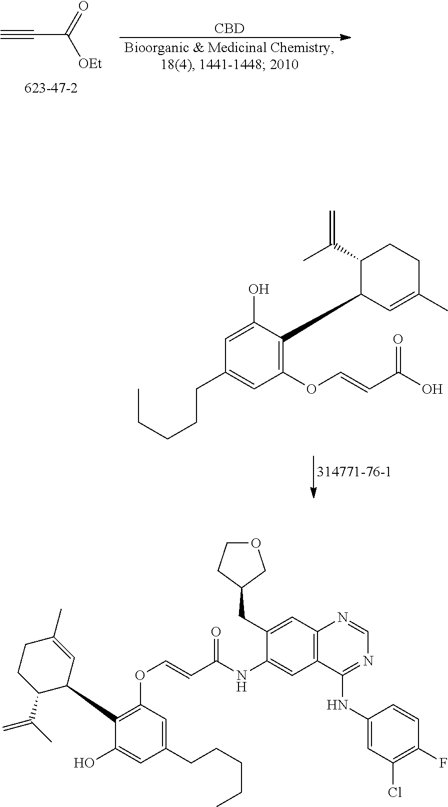

Michael Acceptor vinyl sulfone compounds are prepared from CBD and the building block [13894-21-8] using conditions similar to those referenced in the Scheme below. The double bond isomers may be separated and isolated by chromatography.

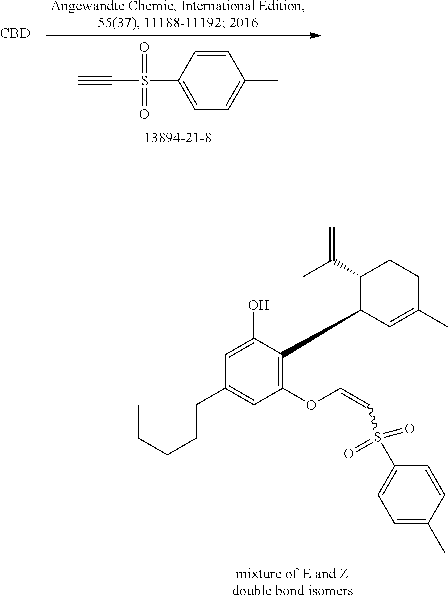

[87] Michael Acceptor vinyl sulfonamide compounds are prepared from a cannabinoid (CBD) and an alkynyl sulfonamide building block, in this case [250583-24-5], using conditions similar to those referenced for the related vinyl sulfones and Michael Acceptor ester compounds described above.

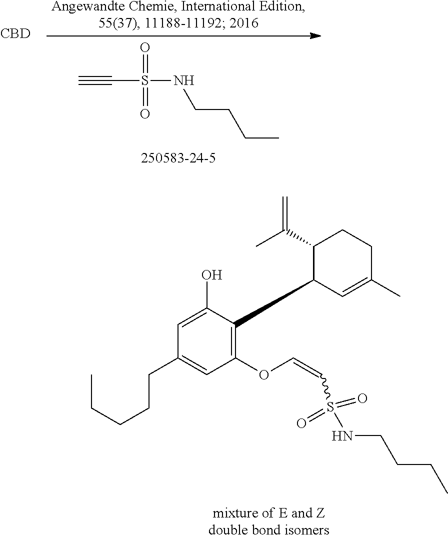

Carbamate conjugate molecules may be synthesized as shown in the scheme below, by reacting a cannabinoid (CBD) with phosgene (or a suitable surrogate) and the appropriate amine building block under standard basic conditions.

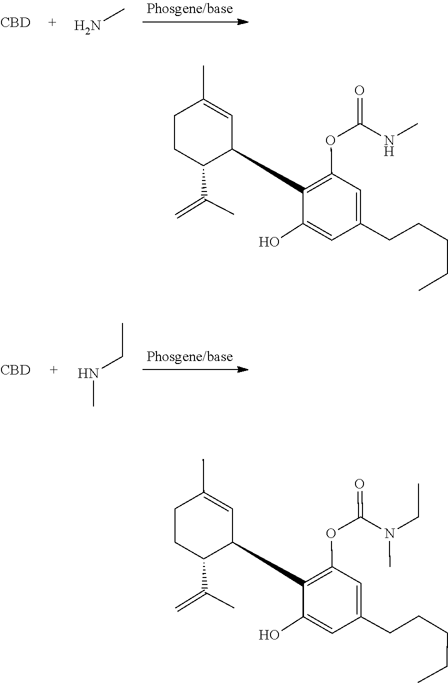

1. A conjugate molecule, or a pharmaceutically acceptable salt thereof, comprising a first therapeutic agent component covalently linked to a first hydroxy or a first carboxylic acid group of a cannabinoid component, wherein the first therapeutic agent component is selected from:
(1) a Michael Acceptor component having a structure selected from 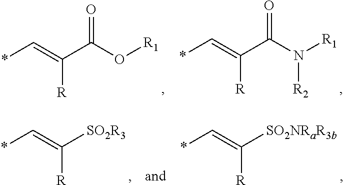
wherein * indicates a site of covalent attachment to the hydroxy group of the cannabinoid component;
(2) 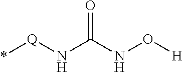
wherein Q is CO, CS, or CR₆R₇; and
(3) a carbamate component having a structure 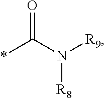
wherein R₈ and R₉ independently are selected from H, CH₃, and CH₂CH₃; and
wherein:
R is selected from the group consisting of:
(a) H;
(b) C1-C8 linear or branched alkyl, optionally substituted with
(1) 1, 2, 3, 4, 5, 6, 7, 8, or 9 fluorine atoms; and/or
(2) 1, 2, or 3 substituents independently selected from the Group One Substituents;
(c) C1-C8 linear or branched heteroalkyl containing 1, 2, or 3 heteroatoms independently selected from O, N, and S and optionally substituted with
(1) 1, 2, 3, 4, 5, 6, 7, 8, or 9 fluorine atoms; and/or
(2) 1, 2, or 3 substituents independently selected from the Group One Substituents;
(d) phenyl, optionally substituted with 1, 2, or 3 substituents independently selected from the group consisting of:
(1) C1-C6 linear or branched alkyl, optionally substituted with
(i) 1, 2, 3, 4, 5, or 6 fluorine atoms; and/or
(ii) 1 or 2 substituents independently selected from the Group Two Substituents; and
(2) C1-C6 linear or branched heteroalkyl containing 1 or 2 heteroatoms independently selected from O, N, and S and optionally substituted with
(i) 1, 2, 3, 4, 5, or 6 fluorine atoms; and/or
(ii) 1 or 2 substituents independently selected from the Group One Substituents;
(e) a 6- to 10-membered aromatic, optionally substituted with 1, 2, 3, or 4 substituents independently selected from the group consisting of:
(1) phenyl;
(2) halide;
(3) cyano;
(4) C1-C6 linear or branched alkyl, optionally substituted with
(i) 1, 2, 3, 4, 5, 6, 7, 8, or 9 fluorine atoms; and/or
(ii) 1, 2, or 3 substituents independently selected from the Group Two Substituents, and
(5) C1-C6 linear or branched heteroalkyl containing 1, 2, or 3 atoms independently selected from O, N, and S and optionally substituted with
(i) 1, 2, 3, 4, 5, 6, 7, 8, or 9 fluorine atoms; and/or
(ii) 1, 2, or 3 substituents independently selected from the Group Two Substituents;
(f) 5- to 10-membered heteroaromatic comprising 1, 2, 3, 4, 5, or 6 heteroatoms independently selected from O, N, and S and optionally substituted with 1, 2, 3, or 4 substituents independently selected from
(1) phenyl;
(2) halide;
(3) cyano;
(4) trifluoromethyl;
(5) C1-C6 linear or branched alkyl optionally substituted with
(i) 1, 2, 3, 4, 5, 6, 7, 8, or 9 fluorine atoms; and/or
(ii) 1, 2, or 3 substituents independently selected from the Group Two Substituents; and
(6) C1-C6 linear or branched heteroalkyl containing 1, 2, or 3 atoms independently selected from O, N, and S and optionally substituted with
(i) 1, 2, 3, 4, 5, 6, 7, 8, or 9 fluorine atoms; and/or
(ii) 1, 2, or 3 substituents independently selected from the Group Two Substituents;
(g) 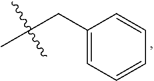 [SMILES: *Cc1ccccc1]
optionally substituted with 1, 2, or 3 substituents independently selected from the group consisting of:
(1) C1-C6 linear or branched alkyl, optionally substituted with
(i) 1, 2, 3, 4, 5, or 6 fluorine atoms; and/or
(ii) 1, 2, or 3 substituents independently selected from the Group Two Substituents;
(h) 3- to 9-membered cycloheteroalkyl having 1, 2, or 3 heteroatoms independently selected from O, N, and S and optionally substituted with 1, 2, or 3 substituents independently selected from the group consisting of:
(1) C1-C6 linear or branched alkyl, optionally substituted with
(i) 1, 2, 3, 4, 5, 6, 7, 8, or 9 fluorine atoms; and/or
(ii) 1, 2, or 3 substituents independently selected from the Group Two Substituents,
(2) C1-C6 linear or branched heteroalkyl, optionally substituted with
(i) 1, 2, 3, 4, 5, 6, 7, 8, or 9 fluorine atoms and/or
(ii) 1, 2, or 3 substituents independently selected from the Group Two Substituents,
(3) phenyl, optionally substituted with 1, 2, or 3 substituents independently selected from the Group Two Substituents, and
(4) 5- to 10-membered heteroaromatic, optionally substituted with 1, 2, or 3 substituents independently selected from the Group Two Substituents; and
(i) C3-C6 cycloalkyl, optionally substituted with 1, 2, or 3 substituents independently selected from:
(1) C1-C6 linear or branched alkyl, optionally substituted with
(i) 1, 2, 3, 4, 5, 6, 7, 8, or 9 fluorine atoms; and/or
(ii) 1, 2, or 3 substituents independently selected from the Group Two Substituents,
(2) C1-C6 linear or branched heteroalkyl, optionally substituted with
(i) 1, 2, 3, 4, 5, 6, 7, 8, or 9 fluorine atoms; and/or
(ii) 1, 2, or 3 substituents independently selected from the Group Two Substituents,
(3) phenyl, optionally substituted with 1, 2, or 3 substituents independently selected from Group Two Substituents; and
(4) 5- to 10-membered heteroaromatic, optionally substituted with 1, 2, or 3 substituents independently selected from the Group Two Substituents;
Group One Substituents is a group of substituents consisting of:
(a) —OH;
(b) —NH₂;
(c) ═O;
(d) ═S;
(e) ═NR7, where R7 is H or is C1-C3 linear or branched alkyl or C1-C3 linear or branched heteroalkyl comprising an O, N, or S atom;
(f) —C(O)OR4, wherein R4 is H or C1-C3 linear or branched alkyl;
(g) —C(O)NR5R6, wherein R5 and R6 independently are H or C1-C6 linear or branched alkyl;
(h) halide;
(i) C1-C6 linear or branched alkoxyl;
(j) C1-C6 linear or branched alkylamino;
(k) C1-C6 linear or branched dialkylamino;
(l) 6- to 10-membered aromatic, optionally substituted with 1, 2, 3, or 4 substituents independently selected from
(i) phenyl;
(ii) halide;
(iii) cyano;
(iv) C1-C6 linear or branched alkyl, optionally substituted with
(1) 1, 2, 3, 4, 5, 6, 7, 8, or 9 fluorine atoms; and/or
(2) 1, 2, or 3 substituents independently selected from the Group Two Substituents; and
(v) C1-C6 linear or branched heteroalkyl containing 1, 2, or 3 atoms independently selected from O, N, and S and optionally substituted with
(1) 1, 2, 3, 4, 5, 6, 7, 8, or 9 fluorine atoms; and/or
(2) 1, 2, or 3 substituents independently selected from the Group Two Substituents;
(m) 5- to 10-membered heteroaromatic, optionally substituted with 1, 2, 3, or 4 substituents independently selected from
(i) phenyl;
(ii) halide;
(iii) cyano;
(iv) C1-C6 linear or branched alkyl, optionally substituted with
(1) 1, 2, 3, 4, 5, 6, 7, 8, or 9 fluorine atoms; and/or
(2) 1, 2, or 3 substituents independently selected from the Group Two Substituents; and
(v) C1-C6 linear or branched heteroalkyl containing 1, 2, or 3 atoms independently selected from O, N, and S and optionally substituted with
(1) 1, 2, 3, 4, 5, 6, 7, 8, or 9 fluorine atoms; and/or
(2) 1, 2, or 3 substituents independently selected from the Group Two Substituents;
(n) 3- to 9-membered cycloheteroalkyl having 1, 2, or 3 heteroatoms independently selected from O, N, and S, optionally substituted with 1, 2, 3, or 4 substituents independently selected from
(i) phenyl;
(ii) halide;
(iii) cyano;
(iv) C1-C6 linear or branched alkyl, optionally substituted with
(1) 1, 2, 3, 4, 5, 6, 7, 8, or 9 fluorine atoms; and/or
(2) 1, 2, or 3 substituents independently selected from the Group Two Substituents; and
(v) C1-C6 linear or branched heteroalkyl containing 1, 2, or 3 atoms independently selected from O, N, and S and optionally substituted with
(1) 1, 2, 3, 4, 5, 6, 7, 8, or 9 fluorine atoms; and/or
(2) 1, 2, or 3 substituents independently selected from the Group Two Substituents; and
(o) C3-C6 cycloalkyl, optionally substituted with 1, 2, 3, or 4 substituents independently selected from
(i) phenyl;
(ii) halide;
(iii) cyano;
(iv) C1-C6 linear or branched alkyl, optionally substituted with
(1) 1, 2, 3, 4, 5, 6, 7, 8, or 9 fluorine atoms; and/or
(2) 1, 2, or 3 substituents independently selected from the Group Two Substituents; and
(v) C1-C6 linear or branched heteroalkyl containing 1, 2, or 3 atoms independently selected from O, N, and S and optionally substituted with
(1) 1, 2, 3, 4, 5, 6, 7, 8, or 9 fluorine atoms; and/or
(2) 1, 2, or 3 substituents independently selected from the Group Two Substituents;
Group Two Substituents is a group of substituents consisting of:
(a) —OH;
(b) —NH₂;
(c) ═O;
(d) ═S;
(e) ═NR7, where R7 is H or is C1-C3 linear or branched alkyl or C1-C3 linear or branched heteroalkyl comprising an O, N, or S atom;
(f) —C(O)OR4, wherein R4 is H or C1-C3 linear or branched alkyl;
(g) —C(O)NR5R6, wherein R5 and R6 independently are H or C1-C6 linear or branched alkyl;
(h) halide;
(i) cyano;
(j) trifluoromethyl;
(k) C1-C6 linear or branched alkoxyl;
(l) C1-C6 linear or branched alkylamino;
(m) C1-C6 linear or branched dialkylamino;
(n) 6- to 10-membered aromatic; and
(o) 5- to 10-membered heteroaromatic comprising 1, 2, 3, 4, 5, or 6 heteroatoms independently selected from O, N, and S;
R₁ and R₂ independently are selected from the group consisting of:
(a) C1-C12 linear or branched alkyl, optionally substituted with
(i) 1, 2, 3, 4, 5, 6, 7, 8, or 9 fluorine atoms; and/or
(ii) 1, 2, or 3 substituents selected from the Group One Substituents;
(b) C2-C12 linear or branched alkenyl, optionally substituted with
(i) 1, 2, 3, 4, 5, 6, 7, 8, or 9 fluorine atoms; and/or
(ii) 1, 2, or 3 substituents selected from the Group One Substituents;
(c) C1-C12 linear or branched heteroalkyl containing 1, 2, 3, or 4 heteroatoms independently selected from O, N, and S, optionally substituted with
(i) 1, 2, 3, 4, 5, 6, 7, 8, or 9 fluorine atoms; and/or
(ii) 1, 2, or 3 substituents selected from the Group One Substituents; and
(d) R; OR
R₁ and R₂, together with the atom to which they are attached, form a 3- to 9-membered cycloheteroalkyl having 1, 2, 3, or 4 heteroatoms independently selected from O, S, and N, wherein the cycloheteroalkyl optionally is substituted with 1, 2, or 3 substituents independently selected from, C1-C6 linear or branched alkyl optionally substituted with 1, 2, 3, 4, 5, 6, 7, 8, or 9 fluorine atoms, C1-C6 linear or branched heteroalkyl optionally substituted with 1, 2, 3, 4, 5, 6, 7, 8, or 9 fluorine atoms, phenyl optionally substituted with 1, 2, or 3 substituents independently selected from Group Two Substituents, or 5- to 10-membered heteroaromatic optionally substituted with 1, 2, or 3 independently selected from Group Two Substituents;
R₃, R₃ₐ, and R_{3b} independently are selected from
(a) C1-C8 linear or branched alkyl, optionally substituted with
(i) 1, 2, 3, 4, 5, 6, 7, 8, or 9 fluorine atoms; and/or
(ii) 1, 2, or 3 substituents independently selected from the Group One Substituents; or
(b) phenyl, optionally substituted with up to three substituents independently selected from the group consisting of C1-C6 linear or branched alkyl, optionally substituted with
(i) 1, 2, 3, 4, 5, or 6 fluorine atoms; and/or
(ii) 1 or 2 substituents independently selected from the Group Two Substituents; and
R₆ and R₇ independently are R.

2. The conjugate molecule of claim 1, wherein the first therapeutic agent component is a Michael acceptor component, and the Michael acceptor component is selected from the group consisting of

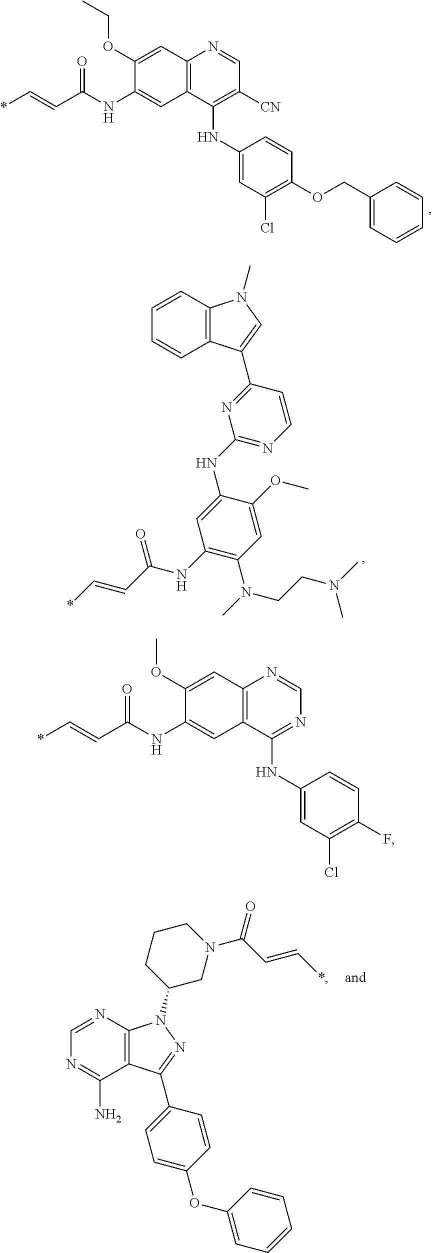

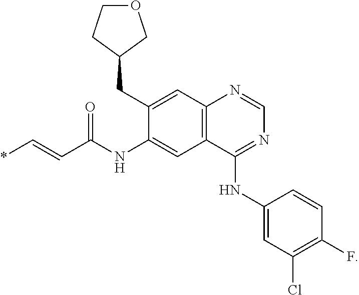 [SMILES: */C=C/C(=O)Nc1cc2c(Nc3ccc(F)c(Cl)c3)ncnc2cc1C[C@H]1CCOC1]

3. The conjugate molecule of claim 1, or the pharmaceutically acceptable salt thereof, wherein the cannabinoid component comprises a first hydroxy group.

4. The conjugate molecule of claim 1, or the pharmaceutically acceptable salt thereof, wherein the cannabinoid component comprises a first carboxylic acid group.

5. The conjugate molecule of claim 3, or the pharmaceutically acceptable salt thereof, wherein the cannabinoid component further comprises a second hydroxy group or a second carboxylic acid group.

6. The conjugate molecule of claim 3, or the pharmaceutically acceptable salt thereof, further comprising a second therapeutic agent component covalently attached to the cannabinoid component.

7. The conjugate molecule of claim 6, or the pharmaceutically acceptable salt thereof, wherein the second therapeutic agent component is:

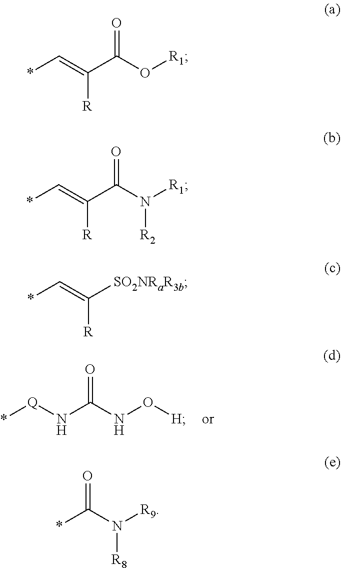

8-12. (canceled)

13. The conjugate molecule of claim 1, or the pharmaceutically acceptable salt thereof, wherein:
(a) the cannabinoid component is provided by a cannabinoid selected from the group consisting of a cannabigerol, a cannabichromene, a cannabidiol, a tetrahydrocannabinol, a cannabicyclol, a cannabielsoin, a cannabinol, a cannabinodiol, a cannabitriol, a dehydrocannabifuran, a cannabifuran, a cannabichromanon, and a cannabiripsol, or an active metabolite thereof, or
(b) wherein the cannabinoid component is a cannabidiol component.

14. (canceled)

15. A pharmaceutical composition comprising a conjugate molecule of claim 1, or a pharmaceutically acceptable salt thereof, and a pharmaceutically acceptable vehicle.

16. The pharmaceutical composition of claim 15, which:
(a) comprises a racemic mixture of conjugate molecules;
(b) comprises a single enantiomer of the conjugate molecule;
(c) comprises a mixture of diastereomers of the conjugate molecule;
(d) comprises a mixture of double bond isomers of the conjugate molecule;
(e) comprises a Z-double bond isomer of the conjugate molecule;
(f) comprises a E-double bond isomer of the conjugate molecule; or
(g) comprises an isotopic variant of the conjugate molecule.

17-21. (canceled)

22. A method of treating a hyperproliferative disorder, comprising administering to a patient in need thereof a conjugate molecule of claim 1 or a pharmaceutically acceptable salt thereof, wherein the therapeutic agent component is selected from the group consisting of

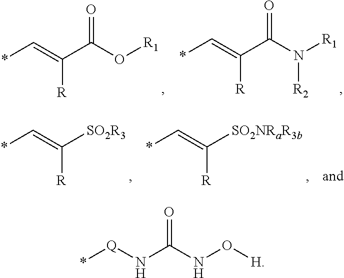

23. The method of claim 22, wherein the hyperproliferative disorder is a cancer.

24. The method of claim 23, wherein the conjugate molecule is administered in conjunction with a second cancer therapy.

25. A method of treating glaucoma or of reversing central or peripheral anticholinergia, comprising administering to a patient in need thereof a conjugate molecule of claim 1, or a pharmaceutically acceptable salt thereof, wherein the therapeutic agent component is a physostigmine-based carbamate component.

26. A method of treating confusion or dementia, comprising administering to a patient in need thereof a conjugate molecule of claim 1, or a pharmaceutically acceptable salt thereof, wherein the therapeutic agent component is a rivastigmine-based carbamate component.
